(19) United States
(12) Patent Application Publication
(10) Pub. No.: US 2023/0225652 A1
(43) Pub. Date: Jul. 20, 2023

(54) METHOD OF EVALUATION OF SOCIAL INTELLIGENCE AND SYSTEM ADOPTING THE METHOD

(71) Applicant: Sangmyung University Industry - Academy Cooperation Foundation, Seoul (KR)

(72) Inventors: Mincheol WHANG, Gyeonggi-do (KR); Gangyoung LEE, Seoul (KR); Hyunwoo LEE, Gyeonggi-do (KR); Ayoung CHO, Gyeonggi-do (KR)

(21) Appl. No.: 18/090,141

(22) Filed: Dec. 28, 2022

(30) Foreign Application Priority Data
Jan. 14, 2022 (KR) 10-2022-0006046

(51) Int. Cl. A61B 5/16; A61B 5/024; G06V 10/764; G06V 40/16

(52) U.S. Cl. CPC A61B 5/165; A61B 5/02405; A61B 5/02416; G06V 10/764; G06V 40/174

(57) ABSTRACT

Provided is a method of evaluating emotional intelligence, the method including evaluating an accuracy of emotional recognition of subjects for the emotional image stimuli, evaluating an accuracy of interpersonal emotional recognition of subjects for a facial image stimulus with a certain emotion, a classification step of presenting a video stimulus of a certain emotion to selected subjects with high accuracy, extracting a heart rate variability (HRV) the subjects, forming a classification model by classification of the video stimulus and machine learning using the HRV through a model generating unit, and evaluating an emotional intelligence of the subject exposed to the image stimulus using the classification model.

### CROSS-REFERENCE TO RELATED APPLICATION

This application is based on and claims priority under 35 U.S.C. §119 to Korean Patent Application No. 10-2022-0006046, filed on Jan. 14, 2022, in the Korean Intellectual Property Office, the disclosure of which is incorporated by reference herein in its entirety.

### BACKGROUND

### 1. Field

The present disclosure relates to a method and apparatus for evaluating social intelligence, and more particularly, to a method and apparatus for evaluating an individual’s emotional intelligence using heart rate variability.

### 2. Description of the Related Art

Emotional intelligence refers to the ability to recognize and control one’s own emotions and the emotions of others to derive the basis for determining the direction of one’s thoughts and actions. The emotional intelligence is very important as a core ability to enjoy life happiness through social success and improvement in human relationships.

Emotions affect not only moods, preferences, and physical states, but also the way we think, make decisions, and do things and this emotional intelligence may effectively cope with countless conflicts and stresses that occur in society and lead this in a positive direction. Emotional intelligence may be improved through education, not through innate ability, so it may be improved through training. Therefore, it is very important to evaluate and take action on the emotional intelligence level during infancy.

### SUMMARY

The inventive concept provides a method and system for evaluating emotional intelligence that may effectively evaluate emotional recognition ability.

The inventive concept also provides a method and system for evaluating emotional intelligence level using heart rate variability (HRV).

Additional aspects will be set forth in part in the description which follows and, in part, will be apparent from the description, or may be learned by practice of the presented embodiments of the disclosure.

According to an aspect of the inventive concept, there is provided a method of evaluating emotional intelligence, the method including
a first evaluation step of presenting emotional image stimuli containing natural or artificial landscapes to a plurality of subjects through a display to evaluate an accuracy of emotional recognition for the emotional image stimuli,
a second evaluation step of presenting a facial image stimulus of a certain emotion to the subject to evaluate an accuracy of interpersonal emotional recognition of the subject,
a subject selection step of classifying subjects with high accuracy and low accuracy among the plurality of subjects according to results of the first evaluation step and the second evaluation step,
a video stimulus classification step of presenting a video of a certain emotion to the subject selected in the above process through the display to classify emotions of the video,
extracting a heart rate variability (HRV) from heartbeat information by a heartbeat information processing unit by extracting the heartbeat information of the subject exposed to a video of a certain emotion in the video stimulus classification step with a sensor,
forming a classification model by classification of the certain video stimulus and machine learning using the HRV through a model generating unit, and
evaluating an emotional intelligence of the subject exposed to the image stimulus using the classification model by an emotional intelligence evaluation unit.

According to one or more embodiments, the certain emotion may be classified into High Arousal and High Valence (HAHV), High Arousal and Low Valence (HALV), Low Arousal and Low Valence (LALV), and Low Arousal and High Valence (LAHV).

According to one or more embodiments, the emotional image stimulus may use a photo stimulus presented by the International Affective Picture System (lAPS).

According to one or more embodiments, the HRV may include a time domain parameter.

According to one or more embodiments, the time domain parameter may include at least one of Heart Rate (HR), Standard Deviation of NN Interval (SDNN), and root Mean Square of successive differences (rMSSD) between intervals between peaks (PPI).

According to one or more embodiments, the HRV may include a frequency domain parameter.

According to one or more embodiments, the frequency domain parameter of the HRV may include at least one of high frequency (HF) power between 0.15 Hz and 0.04 Hz, very low frequency (VLF) power between 0.0033 Hz and 0.04 Hz, low frequency (LF) power between 0.04 Hz and 0.15 Hz, VLF/LF, LF/HF, total power, peak power, dominant power between 0.04 Hz to 0.26 Hz, and coherence ratio (peak power / (total power-peak power)).

According to one or more embodiments, the classification model may be a Super Vector Machine (SVM) classification model.

According to one or more embodiments, the method further includes extracting effective HRV by evaluating a significance of heart rate variability based on an emotional intelligence evaluation score between the extracting of HRV and the forming of a classification model,
wherein the extracting of the effective HRV includes
performing nonparametric one-way analysis of variance (Kruskal-Wallis test) and Bonferroni correction to extract heart rate variability that is significantly different between groups by emotion, wherein for correlation comparison between emotional groups, Kendal-tau correlation analysis is performed to extract heart rate variability, which shows a correlation coefficient of 0.6 or higher.

According to another aspect of the inventive concept, there is provided an emotional intelligence evaluation system including
a display presenting video stimuli with certain emotions to a subject,
a heartbeat information extraction device equipped with a sensor for extracting heartbeat information from the subject,
a heart rate variability (HRV) extraction unit configured to extract HRV from the heartbeat information,
a model formation unit configured to form an emotional intelligence evaluation model using the HRV, and
an emotional intelligence evaluation unit configured to evaluate the subject’s emotional intelligence by applying the HRV of the subject exposed to a certain image stimulus, by using the model obtained from the model formation unit.

According to one or more embodiments, the heartbeat information extraction device may include an electroencephalography (EEG) sensor or a photoplethysmography (PPG) sensor.

According to one or more embodiments, the image stimulus may include a picture stimulus presented by the International Affective Picture System (IAPS).

According to one or more embodiments, the HRV may include a time domain parameter.

According to one or more embodiments, the time domain parameter may include at least one of Heart Rate (HR), Standard Deviation of NN Interval (SDNN), and root Mean Square of successive differences (rMSSD) between intervals between peaks (PPI).

According to one or more embodiments, the HRV may include a frequency domain parameter.

According to one or more embodiments, the frequency domain parameter of the HRV may include at least one of high frequency (HF) power between 0.15 Hz and 0.04 Hz, very low frequency (VLF) power between 0.0033 Hz and 0.04 Hz, low frequency (LF) power between 0.04 Hz and 0.15 Hz, VLF/LF, LF/HF, total power, peak power, dominant power between 0.04 Hz to 0.26 Hz, and coherence ratio (peak power / (total power-peak power)).

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A illustrates a sequence of an emotional intelligence evaluation method according to one or more embodiments;

FIG. 1B illustrates a process of selecting a final photo to be used for emotional intelligence evaluation from candidate images of stimulus images in an emotional intelligence evaluation method according to one or more embodiments;

FIG. 2 illustrates Russell’s two-dimensional emotional expression model;

FIG. 3 illustrates a set of image stimuli of High Arousal and High Valence (HAHV) according to Russell’s two-dimensional emotional classification;

FIG. 4 illustrates a set of High Arousal and Low Valence (HALV) image stimuli according to Russell’s two-dimensional emotional classification;

FIG. 5 illustrates a set of image stimuli of (Low Arousal and Low Valence (LALV) according to Russell’s two-dimensional emotional classification;

FIG. 6 illustrates a set of image stimuli of Low Arousal and High Valence (LAHV) according to Russell’s two-dimensional emotional classification.

FIG. 7 illustrates a process of producing a stimulus image or an image stimulus according to an embodiment of the present disclosure;

FIG. 8A illustrates the arrangement of AUs defined in each part of the face region;

FIG. 8B illustrates combinations of corresponding AUs for each emotion;

FIGS. 9A to 9D illustrate facial expression stimuli for the above four emotions;

FIG. 10A illustrates a flow of an intensity normalization process of a facial image according to an embodiment of the present disclosure;

FIG. 10B shows an evaluation method for classification ability of facial expressions. according to an embodiment of the present disclosure;

FIG. 11 illustrates a process of presenting an image stimulus to a subject according to an embodiment of the present disclosure;

FIG. 12 illustrates videos for emotional stimulation;

FIG. 13 illustrates a process of producing a video for emotional stimulation according to an embodiment of the present disclosure;

FIG. 14 illustrates a process of producing a video that does not induce emotion according to an embodiment of the present disclosure;

FIG. 15A illustrates a process of determining an emotional intelligence classification criterion according to an embodiment of the present disclosure;

FIG. 15B is a graph illustrating classification of emotional intelligence;

FIG. 16 illustrates a process of measuring HRV from a subject while presenting a video stimulus according to an embodiment of the present disclosure, and evaluating valence and arousal by subjective evaluation; and

FIG. 17 illustrates a process for extracting heart rate variability according to an embodiment of the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, embodiments of the concept of the inventive concept will be described in detail with reference to the accompanying drawings. However, the embodiments of the inventive concept may be modified in various other forms, and the scope of the inventive concept should not be construed as being limited due to the embodiments described below. The embodiments of the inventive concept are preferably interpreted as being provided to more completely explain the inventive concept to those of ordinary skill in the art. The same symbols refer to the same elements from time to time. Furthermore, various elements and regions in the drawings are schematically drawn. Accordingly, the inventive concept is not limited by the relative size or spacing drawn in the accompanying drawings.

Terms such as first, second, etc. may be used to describe various elements, but the elements are not limited by the terms. The terms are used only for the purpose of distinguishing one component from another. For example, without departing from the scope of the inventive concept, a first component may be referred to as a second component, and conversely, a second component may be referred to as a first component.

The terms used in the present application are only used to describe certain embodiments, and are not intended to limit the inventive concept. The terms of a singular form may include plural forms unless otherwise specified. In this application, it will be understood that expressions such as “comprising” or “having” are intended to designate that a feature, number, step, operation, component, part, or combination thereof described in the specification exists, and do not preclude in advance the possibility of the existence or addition of one or more other features or numbers, operations, components, parts, or combinations thereof.

Unless defined otherwise, all terms used herein have the same meaning as commonly understood by one of ordinary skill in the art to which the inventive concept belongs, including technical and scientific terms. Also, commonly used terms as defined in advance should be construed to have a meaning consistent with what they mean in the context of the relevant technology, and should not be construed in an overly formal sense unless explicitly defined herein.

When a certain embodiment may be implemented differently, a particular process order may be performed differently from the described order. For example, two processes described in succession may be performed substantially simultaneously, or may be performed in an order opposite to the described order.

Hereinafter, a method and apparatus for evaluating emotional intelligence using heart rate variability will be described in detail according to one or more embodiments.

Heart rate variability as a parameter applied to emotional intelligence evaluation according to the inventive concept is an objective indicator that cannot be consciously controlled and may be utilized in the field of emotional recognition and evaluation research. The heart rate variability may estimate the body homeostasis control mechanism by analyzing the activity of the parasympathetic and sympathetic nerves, which are responses of the autonomic nervous system, and evaluate the physiological balance. According to the polyvagal theory, parasympathetic homeostasis as measured by heart rate variability is related to emotion, attachment, communication, and ability to regulate emotion. That is, it may be evaluated that the higher the parasympathetic homeostasis, the higher the emotional intelligence.

The presentation of the emotional stimulus is to evaluate how accurately the subject may recognize the intended emotion when he or she is exposed to a certain emotional stimulus.

The level of an individual’s unique emotional intelligence is different for each individual, and therefore, some subjects may feel and respond to a certain emotional stimulus that many people feel in common. In other words, a person with high emotional intelligence will choose the emotion that matches the emotions responded to by a large number of people to a certain emotional stimulus, and a person with low emotional intelligence will choose an emotion that is different from most common emotions. The present disclosure provides a method for effectively evaluating or determining individual emotional intelligence, which is based on such individual emotional differences.

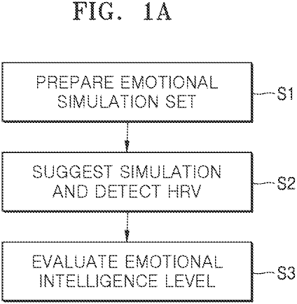

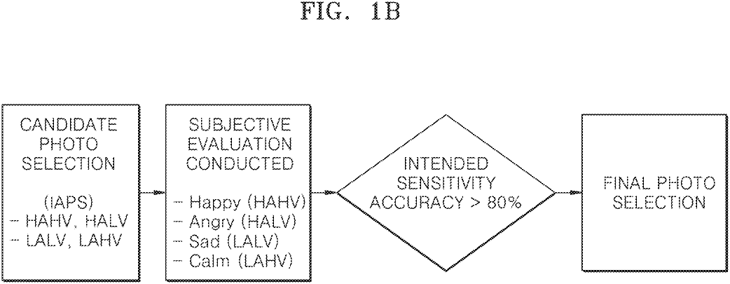

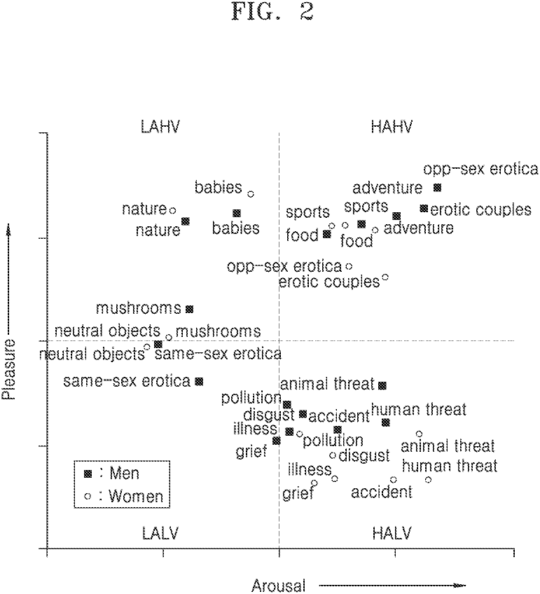

The emotional intelligence evaluation method according to this embodiment includes the following three steps as shown in FIG. 1, and an apparatus for performing this is equipped with hardware and software for performing this method. Basically, the apparatus includes a sensor such as an electrocardiogram (ECG) or a photoplethysmography (PPG) that detects a heartbeat, a heartbeat signal processing unit that processes and analyzes a signal from the sensor, a display that presents an image stimulus to a subject, an image stimulus generating unit that forms the image stimulus, a model generating unit that generates a trained model corresponding to a heartbeat signal from a subject, and an emotional intelligence evaluation unit that evaluates the subject’s emotional intelligence based on a newly input heartbeat signal using the model.

Such a system is based on a computer, and may include a heartbeat signal detection device, a display, an input device such as a keyboard/mouse, and an audio output device for transmitting sound as peripheral devices.
I. Generation of stimuli for assessment of social intelligence
II. Stimulus presentation and heart rate variability extraction
III. Determination of emotional intelligence level

Hereinafter, each step will be described in detail.

### I. Emotional Intelligence Evaluation Based on Emotional Recognition Ability

To produce a stimulus set to be applied in this embodiment, a subject that has passed the following 2 processes is selected.
1) Evaluation of the subject’s emotional and emotional recognition ability for emotional stimuli that contain emotional messages in various environments or atmospheres
2) The subject’s interpersonal emotional recognition ability evaluation (interpersonal empathy ability evaluation) for the emotions expressed in the facial expressions of others is included.

Through the above process, an emotional evaluator for a video for emotional stimulation, which will be described later, is selected from a plurality of subject groups.

### I.1 Emotion Recognition Ability Evaluation

In this step, emotional image stimuli are used, through which the emotional recognition ability of the subject is evaluated. That is, it is evaluated how accurately the subject may accurately classify the intended emotion when exposed to a certain emotional stimulus.

Because each subject has different levels of unique emotional intelligence, there is a possibility that most people will be classified as different emotions by feeling different emotions for the same classified emotional stimulus images. A person with high emotional intelligence will be classified as an emotion that matches the main emotion most selected and classified for a certain emotional stimulus, and a person with low emotional intelligence will be classified as an emotional state different from the emotions most classified.

### I.1.1 Selection of Emotional Stimuli

Candidates for image stimulation that may be used in this step may include landscape photos showing various atmospheric nature, artificial structures photos in which various emotions are expressed, and environmental photos in which certain atmospheres are expressed. As such an emotional image stimulus, a photographic stimulus presented by the International Affective Picture System (IAPS), which is representatively objectified, may be used as an emotional stimulus candidate.

The emotional image stimuli as described above are classified for each emotion by subjective evaluation by a plurality of subjects as evaluation targets.

This classification selects emotions that may represent four quadrants in Russell’s two-dimensional emotional expression model, and there are four categories including High Arousal and High Valence (HAHV), High Arousal and Low Valence (HALV), Low Arousal and Low Valence (LALV), and Low Arousal and High Valence (LAHV).

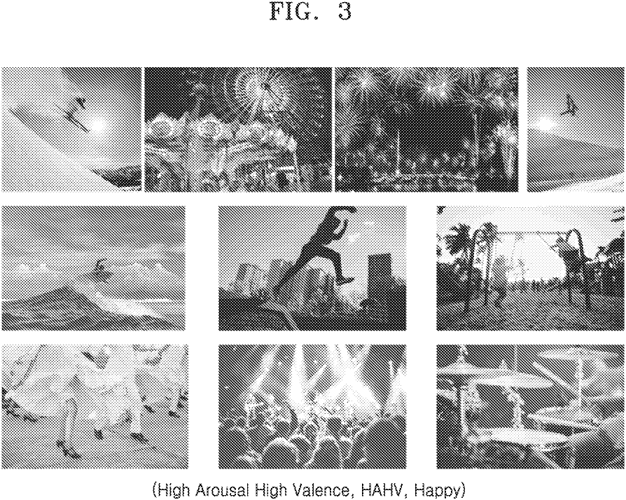

In subjective evaluation, candidate images were randomly presented to the recruited subjects to select a clearer image among candidate images for emotional and emotional stimulation and as a subjective evaluation, one of the four emotions (Happy, Anger, Sad, and Calm), which is the target emotion, was selected. Here, Happy stands for HAHV, Angry stands for HALV, Sad stands for LALV, and Calm stands for LAHV. After the end of the experiment, a total of 40 images were selected, each having an accuracy of more than 80% in selecting the target emotion, and candidate images used in this case are as shown in FIGS. 3 to 6.

### I.1.2 Evaluation of Emotional Recognition Ability Using Emotional Stimuli

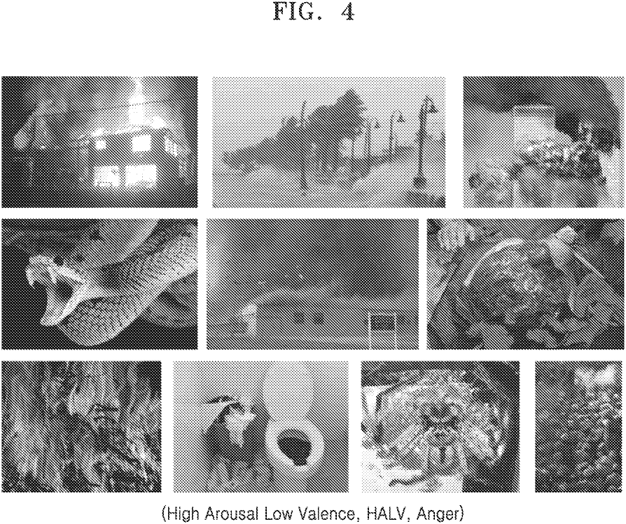

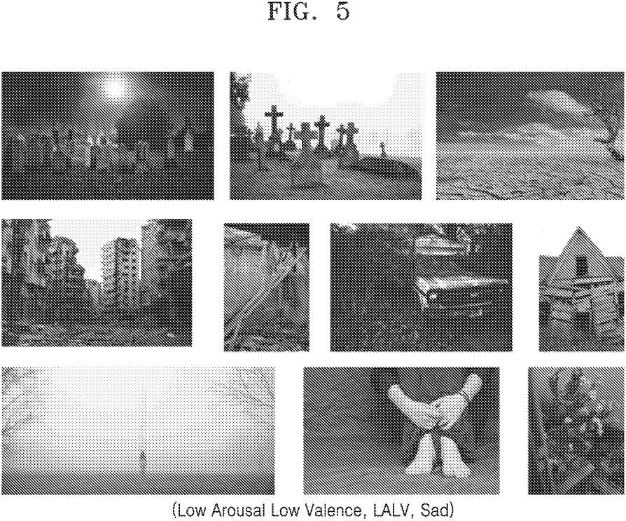

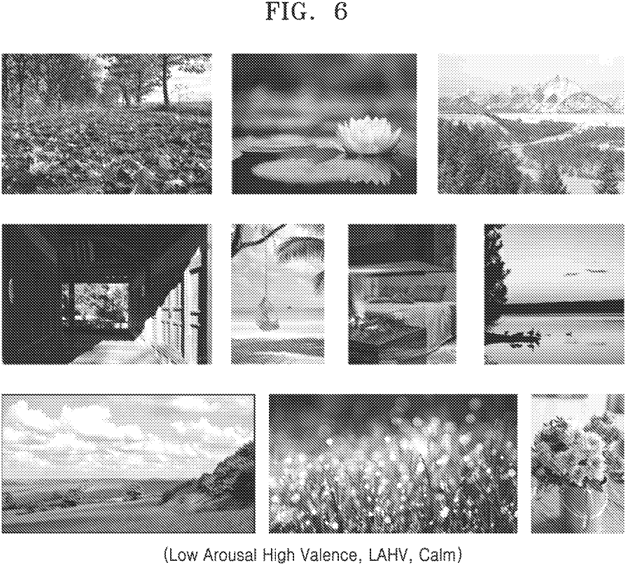

The emotional intelligence of the emotional stimulus evaluator was evaluated using the emotional stimulus selected in the above process. From the above image sets, 40 emotional stimulation images were randomly presented to the subject, and the emotions they felt were selected from among the four items (Happy, Anger, Sad, and Calm). Happy is HAHV, Anger is HALV, Sad is LALV, and Calm is LAHV, and if the emotion recognized by the subject matches the defined emotion with respect to the pre-classified (defined) emotion of the presented image stimulus, a preset score, for example, 10 points, is given, such that a total score of 400 was given for the evaluation of the entire image stimulus. FIG. 3 shows a set of image stimuli classified as High Arousal and High Valence (HAHV), FIG. 4 shows a set of image stimuli classified as High Arousal and Low Valence (HALV), FIG. 5 shows a set of image stimuli classified as Low Arousal and Low Valence (LALV), and FIG. 6 shows a set of image stimuli classified as Low Arousal and High Valence (LAHV).

### I.2 Interpersonal Emotional Recognition (Empathy) Ability Evaluation

### I.2.1 Stimulus Production for Interpersonal Emotional Evaluation

Facial expression images are objective stimuli for evaluating the subject’s ability to recognize facial expressions expressed by others.

As the facial expression image stimulation, a live-action facial image or an artificial facial image may be used. Here, in the case of a live-action facial image, there may be differences in facial expressions for each emotion that are different for each individual, and therefore, it is necessary to make a general facial expression that everyone may sympathize with for each emotion when shooting a live-action facial image for testing. In the case of such an artificial face image, for example, an avatar face image, it is easy to accurately implement a general facial expression. In the implementation of the avatar’s facial image, if the change of expression is possible based on an action unit (AU) defined on the face, this change follows the definition of the facial expression of Ekman’s Facial Action Coding System (FACS).

The creation of an avatar that expresses emotions through the movement of an AU based on FACS will be described.

Human emotions are generally performed through facial expressions, and therefore, in this embodiment, a facial expression stimulation image is produced for interpersonal emotional empathy evaluation. The types of emotions expressed here are the same as before, with a total of 4 types, such as HAHV, HALV, LALV, and LAHV, and Ekman’s FACS and Russell’s Circumplex Model of Affect are referenced to produce facial expression stimulation images.

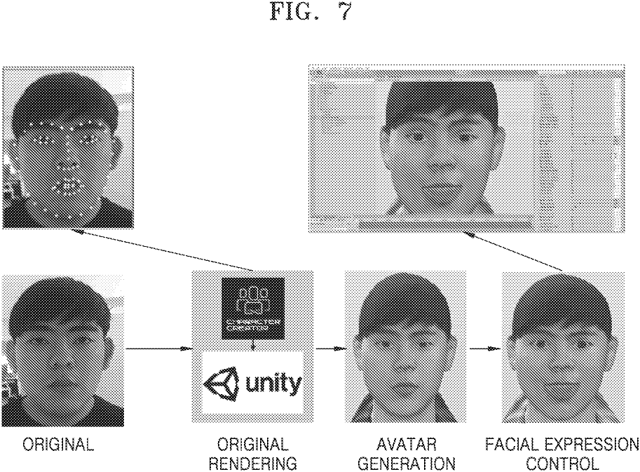

The facial expression stimulus image reflects the representativeness of the facial expression and has a normalized facial expression intensity. The production of such a stimulus image goes through a process as shown in FIG. 7, and here, a tool for creating an avatar image, for example, Character Creator and Unity, which changes facial expressions by giving the AU, may be used.

The Character Creator renders the avatar from the original facial image, and Unity gives the AU of the rendered avatar and manipulates the AU parameter to create an image with changed facial expressions for each of the four emotions, such as Happy, Anger, Sad, and Calm.

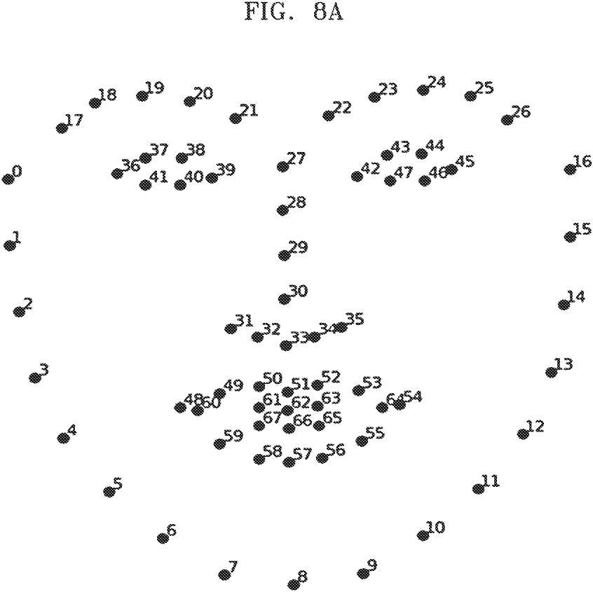

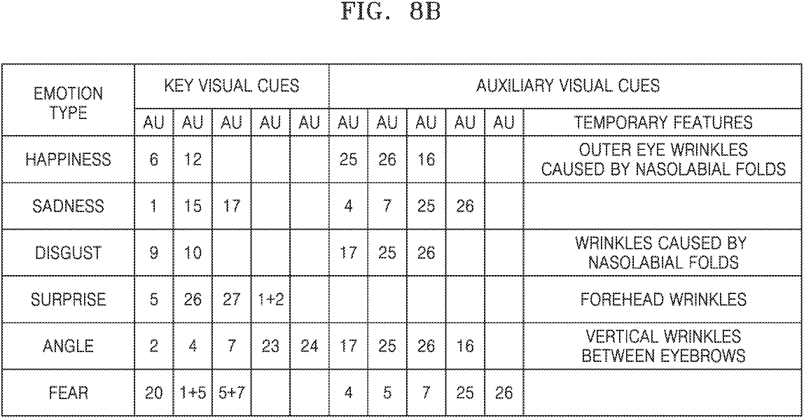

FIG. 7 shows the arrangement of AUs in the facial part defined by the FACS, and FIG. 8B is a table showing combinations of AUs according to expressions such as happiness, sadness, disgust, surprise, anger, and fear.

As described above, it is also possible to individually create an expression intensity change image in which the depth or intensity of facial expression increases by Unity as a single still cut for each intensity but it is possible to produce a facial expression image in the form of a video in which the expression of the corresponding intensity is maintained at a certain intensity level.

In producing a video stimulus in the form of a video, the facial expression stimulus image may be produced so that, for example, an expression change time of 900 msec and an expression maintenance of 100 msec are made.

### I.2.2 Intensity Normalization of Facial Images

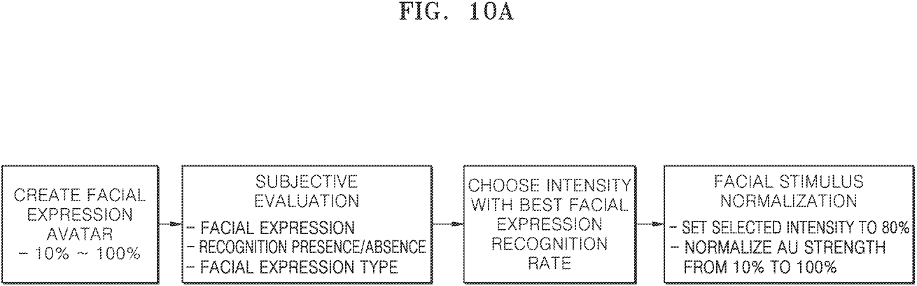

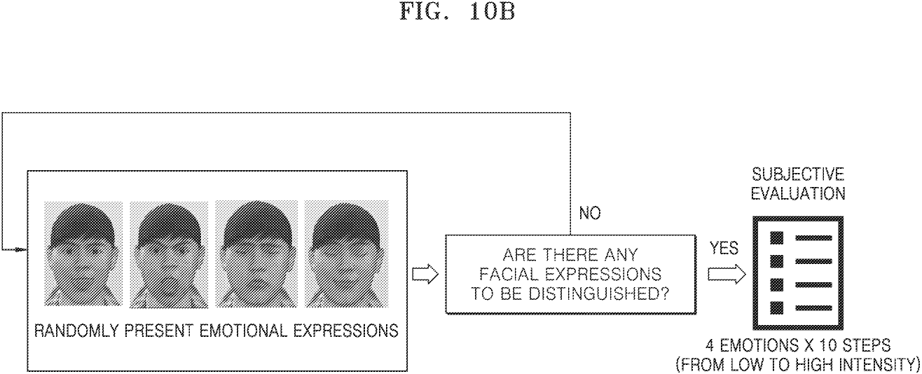

There is a need to standardize facial expression intensity to standardize the scores of facial expressions for the four emotions, such as happiness, anger, sadness, and calm. FIG. 10A shows the flow of the intensity normalization process of a facial image.

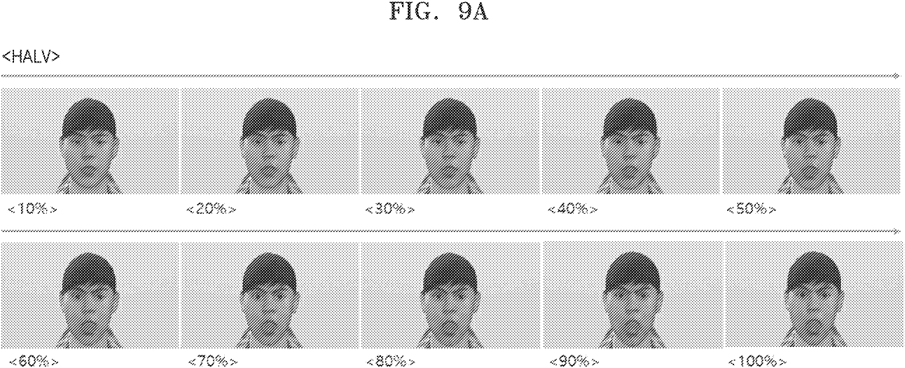

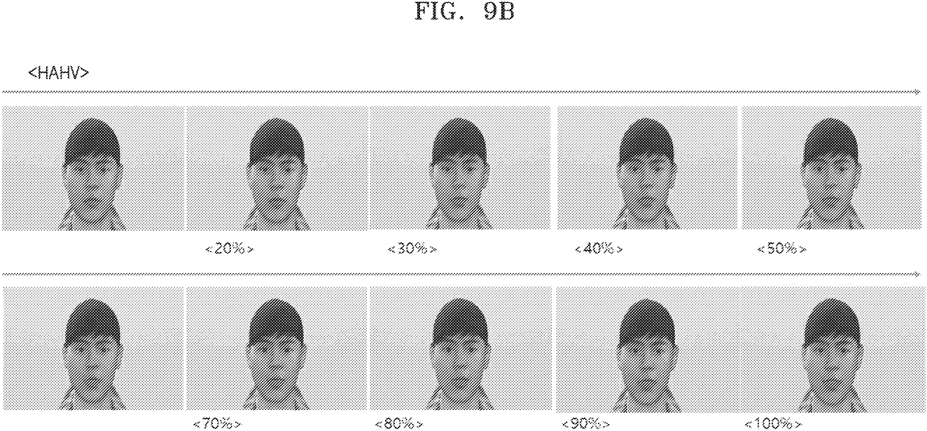

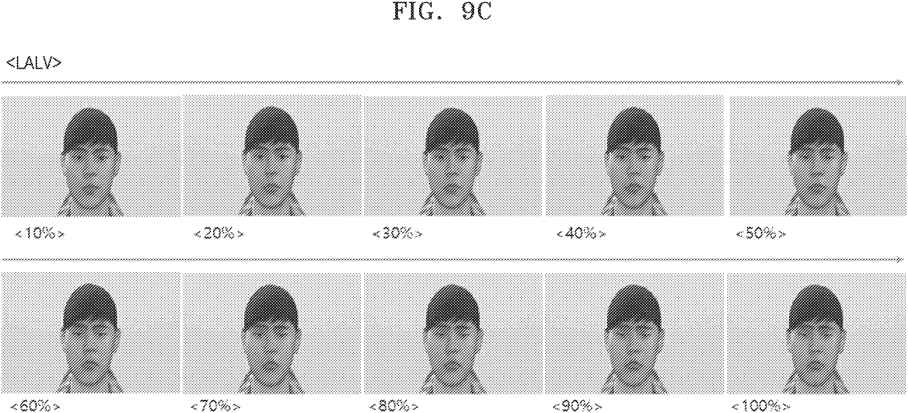

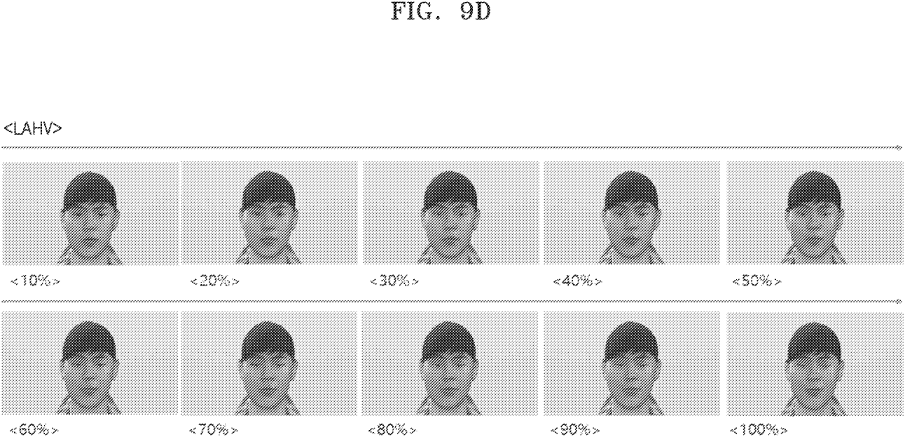

The maximum stimulus intensity is set to 100, which is the maximum value of the parameter provided by Unity, and is reduced by 10% to produce a total of 10 steps (FIGS. 9A to 9D). Subjects were randomly presented with images and asked to select one of four emotions, such as Happy, Anger, Sad, and Calm.

The facial expression stimulation image showing the highest accuracy is set at 80%, which is the intensity at which the intensity recognition accuracy no longer varies between women and men, and then the facial expression intensity is corrected in 10 steps (10% to 100%).

FIGS. 9A to 9D show the facial expression stimuli for the four emotions, and it may be seen that the stimulation of each emotion is increased in intensity by 10%, and therefore the facial expressions are expressed more and more strongly.

### I.2.3 Interpersonal Emotional Recognition Ability Evaluation

In this step, stimulus presentation is made to evaluate how sensitive the subject may recognize various intensity stimuli of facial expressions.

Interpersonal emotion recognition ability evaluation is a step to evaluate how accurately the subject classifies the emotions of each facial expression when looking at various facial expressions.

People with high emotional intelligence may classify the emotions of facial expressions well, but people with low emotional intelligence will not be able to classify the emotions of facial expressions well. In this step, the facial expression stimulus image is repeatedly reproduced. FIG. 10B shows an evaluation method for classification ability of facial expressions. In this step, four types of facial expression stimuli of the same intensity and image stimuli such as happiness, anger, sadness, and calmness of the same expression intensity are presented side by side at the same time. The subject is asked to compare each image and choose which emotion it is. The number of image stimuli before is 40, and if the recognized emotion and the defined emotion match, 10 points are given, and the total score is 400 points.

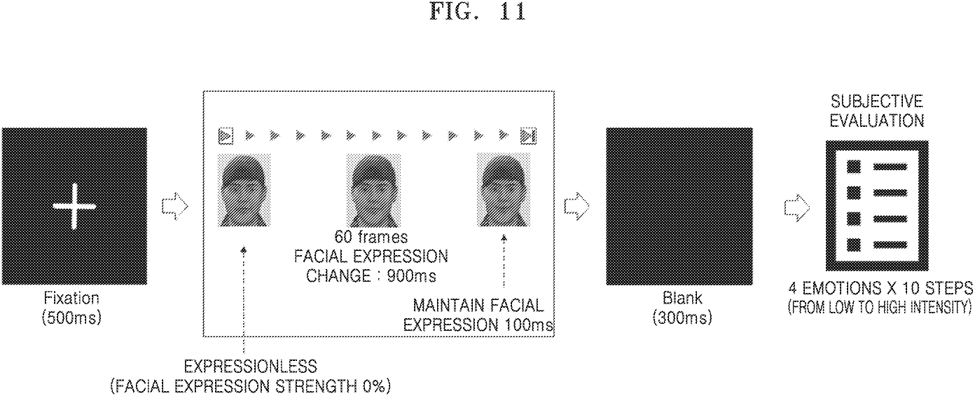

There is a difference in individual perception according to the intensity of emotional expression appearing on the face. That is, a person with high emotional intelligence may recognize low intensity facial expressions well, but a person with low emotional intelligence will not be able to recognize high intensity facial expressions well. FIG. 11 shows a process of presenting an image stimulus to a subject. In this step, as shown in FIG. 11, the facial expression stimulation image is reproduced only once, and a 500 msec black screen (Fixation) in front of the stimulation image and a 300 msec black screen (Blank) are presented after the stimulation image to limit the exposure time of the stimulation image. One type of facial expression stimulus is presented in ascending order from low intensity, that is, increasing the intensity of the emotion by 10%. In this state, the subject was asked to select the recognized emotion from four items, such as Happy, Anger, Sad, and Calm along with whether the facial expression was recognized. At this time, the total number of units of the visual image was 40, and if the recognized emotion and the defined emotion were the same, 10 points were given, giving a total of up to 400 points.

The scores from one emotional image stimulation and two interpersonal image stimulation tests performed above are summed to obtain an average, and this is applied as an emotional intelligence score, and based on the evaluation, an appraiser or classifier suitable for the evaluation of the emotional stimulus may be selected.

### II. Stimulus Presentation and Heart Rate Variability Extraction

According to the polyvagal theory, people with superior emotional regulation ability have better parasympathetic homeostasis than those with low ability to maintain their body from external stimuli. Emotional control ability is a factor that evaluates emotional intelligence, and people with high emotional intelligence have higher emotional control ability than people with low emotional intelligence. In other words, it may be said that people with excellent emotional control ability have high emotional intelligence and have high parasympathetic homeostasis. Parasympathetic homeostasis may be confirmed by measuring heart rate variability.

Since there will be differences between heart rate variability according to emotional intelligence level during emotional stimulation, heart rate variability is extracted during emotional stimulation to confirm the difference.

To evaluate emotional intelligence with heart rate variability, it is measured by electrocardiogram (ECG) or photoplethysmography (PPG) while the subject watches emotionally evoking images.

### II.1. Emotional Stimulation Video

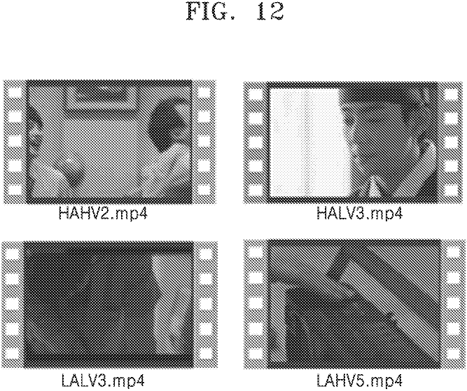

As described above, as for the emotion-induced stimulation image, one video that stimulates the emotions of four types, such as HAHV, HALV, LAHV, and LALV, is prepared. FIG. 12 illustrates videos for emotional stimulation.

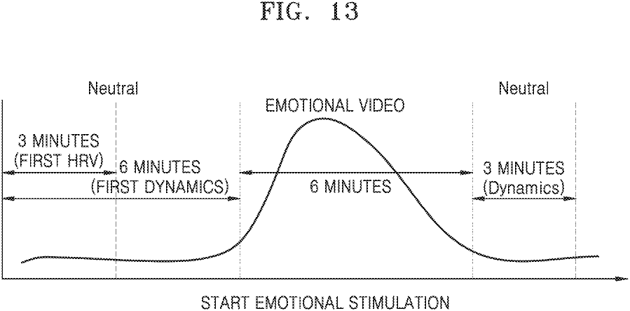

In producing a general-purpose emotion-evoking stimulus image to be used in practice, for the emotion-inducing stimulus image, an image with a high facial expression exposure frequency corresponding to the target emotion classified into HAHV, HALV, LAHV, LALV, etc. is selected. To focus the subject’s attention on the stimulus image, it is necessary to edit and remove scenes that are not related to the event. As shown in FIG. 13, the stimulation image that induces emotion is organized to be at least 6 minutes or longer and a neutral image that does not induce emotion is added, consisting of 6 minutes (first dynamics) in front of the stimulus image that induces emotion, and 3 minutes (dynamics) after the stimulus image.

In selecting an emotionally evoking stimulus image, subjects were asked to select a subjective feeling within 7 scales of excitability (very calm[0] - very excited[7]), and valence (very negative[0] - very positive[7]) as to how much emotion they felt about the emotionally evoking stimulus image, and the average of excitement and valence is derived. For each emotion, a total of four images are selected, one for the most extreme of the target emotion.

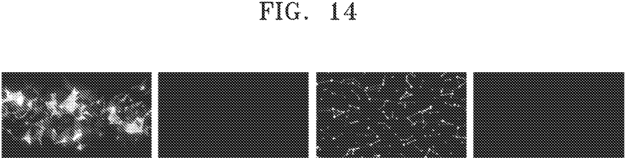

In producing a neutral image that does not induce emotion, an atypical image that does not include a general object is selected and processed in black and white to block emotion induction as shown in FIG. 14.

### II.2. Selection of Emotional Stimulation Video

In the process of selecting video stimuli, candidate images collected through the tests of emotional and emotional stimuli and interpersonal image stimuli in the previous process are presented to selected subjects, and then each subject evaluates their own emotions felt after seeing the video stimuli.

As evaluation responses, responses are given in terms of valence [very negative (1) - very positive (7)] and arousal [very relaxed (1) - very excited (7)]. One video is selected for each emotion, in which the average of subjective evaluation valence and arousal level most closely matches the target emotion.

### II.3. Heart Rate Variability Detection

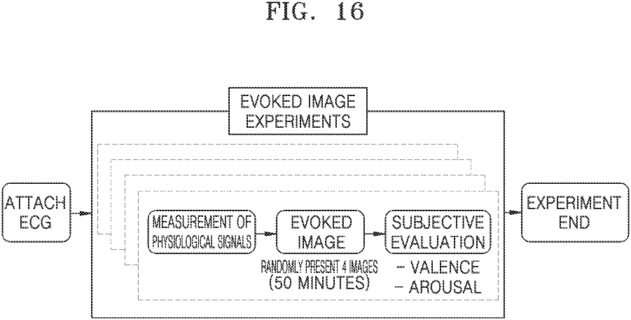

FIG. 16 shows a process of measuring HRV from a subject while presenting a video stimulus, and evaluating valence and arousal by subjective evaluation.

As shown in FIG. 16, HRV extraction is performed in a state where an image stimulus is presented to the subject.

Among subjects, subjects who have already been exposed to a stimulus once may not be able to induce emotion well, so the subject participating in the heart rate variability extraction process may be different from the subjects recruited for stimulus selection.

To extract heart rate variability, electrocardiogram (ECG) signals were measured using an electrocardiogram detection device, Biopac MP100 system (Biopac System, Inc., USA), and may be digitized through an ECG signal preprocessor, for example, the LabView 2014 (National Instruments Corporation, USA) program. ECG measurement methods are possible in various ways. Two types of signals, Time Domain and Frequency Domain, are extracted from heart rate variability from ECG signals. As shown in Tables 1 and 2, the time domain signals are calculated as Peak to Peak, and the frequency domain signals are obtained by Fast Fourier (FFT) for converting to a frequency band.

The time domain parameter of the HRV may include a heart rate (HR), a standard deviation of NN interval (SDNN), a root mean square of successive differences (rMSSD), and a respiratory sinus arrhythmia (RSA).

The parameters of the frequency domain of the HRV may include high frequency (HF) power between 0.15 Hz and 0.04 Hz, very low frequency (VLF) power between 0.0033 Hz and 0.04 Hz, low frequency (LF) power between 0.04 Hz and 0.15 Hz, VLF/LF, LF/HF, total power, peak power, dominant power between 0.04 Hz to 0.26 Hz, and coherence ratio (peak power / (total power-peak power)).

TABLE 1

| | | | |
|---|---|---|---|
| Feature | | Meaning | Calculation |
| Time Domain | BPM | Mean of heart rate in beats per minute | $M e a n \left(P P I\right) = \frac{1}{2} {\sum }_{i = 1}^{N} O P P {I}_{i}$ |
| | SDNN | Standard deviation of all peak to peak intervals | $\sqrt{\frac{1}{N - 1} {\sum }_{i = 1}^{N} {\left[M e a n \left(P P I\right) - P P {I}_{i}\right]}^{2}}$ |
| | rMSSD | Root mean square of successive differences between peak to peak intervals | $\sqrt{\frac{1}{N - 1} {\sum }_{i = 2}^{N} {\left(P P {I}_{i} - P P {I}_{i - 1}\right)}^{2}}$ |
| | RSA | Shortened during inspiration and prolonged during expiration between Peak to Peak interval | $a b s \left(\frac{1}{n} {\sum }_{i = 1}^{N} l n \left(v a r {\left(P P {I}_{i} - \overline{P P I}\right)}^{2}\right)\right)$ |

TABLE 2

| | | | |
|---|---|---|---|
| Feature | | Meaning | Calculation |
| Frequency Domain | VLF | Comprised of rhythms within periods between 25 and 300 s | ${\sum }_{i = \frac{0.0033}{d f}}^{\frac{0.04}{d f}} P o w e {r}_{i}$ |
| | LF | Comprised of rhythms within periods between 7 and 25 s | ${\sum }_{i = \frac{0.04}{d f}}^{\frac{0.15}{d f}} P o w e {r}_{i}$ |
| | HF | Comprised of rhythms within periods between 9 and 24 s | ${\sum }_{i = \frac{0.15}{d f}}^{\frac{0.4}{d f}} P o w e {r}_{i}$ |
| | LF/HF | LF divided by HF | $\frac{L F}{H F}$ |
| | Total Power | Sum of the energy in the VLF, LF and HF | ${\sum }_{i = \frac{0.0033}{d f}}^{\frac{0.04}{d f}} P o w e {r}_{i}$ |

In Table 2 above, distance of frequency (df) is the value obtained by dividing the average of Peak to Peak from 0.5.

The above time domain and frequency domain parameters will be described in more detail as follows.

### I. Time Domain of HRV

In the time domain of HRV, three dynamic characteristic parameters are extracted as follows.
Heart rate (HR): the number of heartbeats per minute obtained from an interval between peaks (PPI)
Standard deviation of NN interval (SDNN): standard deviation of all peaks and the PPI
Percentage of NN interval over 50 s (pNN50): the percentage of the difference in PPI greater than 50 msec (%)

### II. Frequency Domain of HRV

In the frequency domain of HRV, eight dynamic characteristic parameters are extracted as follows. When extracting the frequency domain HRV, the previously extracted PPI is resampled with a frequency of 2 Hz, then the power spectrum (PSD) is calculated using the FFT, and then the following variables are extracted.
Very low frequency (VLF): log transformation value (InVLF) of power value corresponding to 0.0033 Hz to 0.04 Hz bands
Low frequency (LF): log transformation value (InLF) of the power value corresponding to 0.04 Hz to 0.15 Hz bands
VLF/LF: ratio of VLF power to LF power without log transformation applied
LF/HF: ratio of LF power to HF power without log transformation applied
Total power tPow: the entire power spectrum band between 0.0033 4 Hz and 0.4 Hz
Peak power pPow: the power value of the highest peak in the power spectrum
Dominant power dPow: the power spectrum band between -0.015 Hz and 0.015 Hz centered on the peak Hz (peak Hz: the frequency value of the highest peak in the power spectrum band between 0.04 Hz and 0.26 Hz
Coherence ratio CohRatio: Peak power / (total power-peak power)

### III. Time Domain of RSA

To measure RSA, various dynamic characteristic parameters may be used. Accordingly, in this embodiment, three types of parameters, such as the Porges-Bohrer method RSA_PB, rMSSD, and HF as a frequency domain parameter, are measured as dynamic characteristic parameters of the RSA as a time domain parameter. Here, since rMSSD and HF may be affected by the average heartbeat, average PPI normalization and log transformation may be applied together.
RSA_PB: the method proposed by Porges-Bohrer is extracted in the following way.

a. After PPI 2 Hz resampling, noise is removed through 21 point cubic spline polynomial filter (3 order).

b. Use a low pass filter to remove low frequency bands (0.0095 Hz or less) that may act as noise.

c. After that, the range of the band (0.12 Hz to 0.4 Hz) that matches the breathing frequency band is extracted using BPF.

d. After cutting the extracted signal into a 30-second window, log transformation is performed. The average for each window is derived as the final RSA.
rMSSD: the square root of the average of the sum of the squares of the difference between the peaks. In this example, PPI normalization was performed to minimize the effect due to the basic heartbeat level. In this process, rMSSD / mean PPI was calculated, and log transformation was performed on the result value.
HF: the normalized value of the section corresponding to 0.15~0.04 Hz in the power spectrum (PSD) obtained in the previous process. In this embodiment, PPI normalization was performed to minimize the effect due to the basic heartbeat level. For normalization, HF power/mean PPI was calculated, and log transformation was performed on the results.

### III. Emotional Intelligence Level Determination Step

The step of evaluating the emotional intelligence level based on the measured heart rate variability may be divided into two sub-tasks: 1. heart rate variability selection step based on emotional intelligence evaluation score, and 2. emotional intelligence classification criteria determination step.

### III.1. Emotional Intelligence Classification Criteria Determination Step

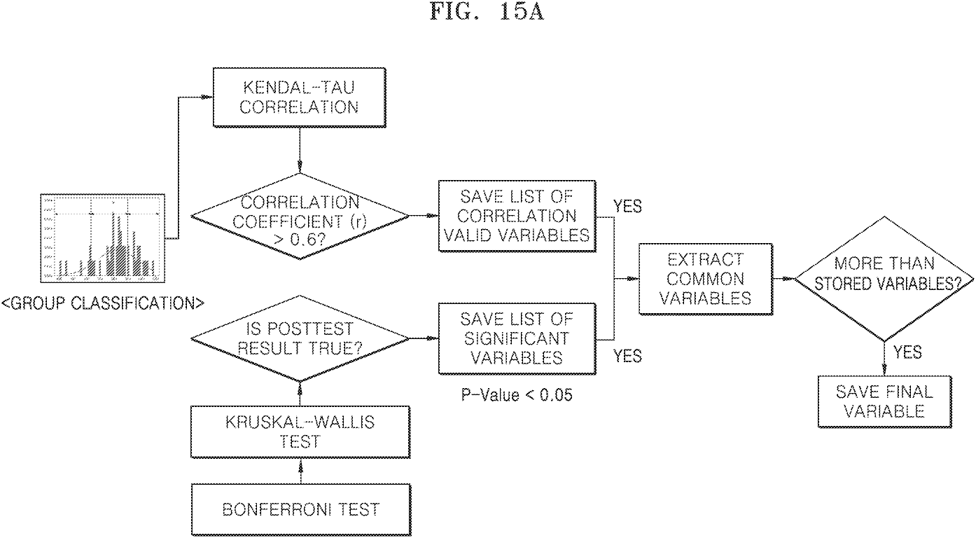

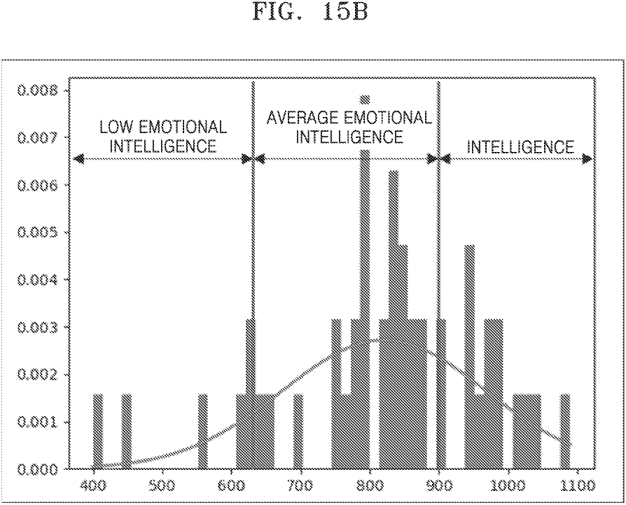

FIG. 15A illustrates a process of determining emotional intelligence classification criteria. In selecting heart rate variability based on emotional intelligence evaluation score, in the emotional intelligence evaluation score-based heart rate variability comparison step, the emotional intelligence evaluation scores are sorted in ascending order, and as shown in FIG. 15B, three groups (low emotional intelligence, average emotional intelligence, high emotional intelligence) are classified by the number of cases and compared.

Kendall’s Tau and Kruskal-Wallis test are used to derive effective variables among heart rate variability, and FIG. 15A shows the process of extracting effective variables according to these statistical techniques. The minimum number of people for emotional intelligence group classification is 5% or more of the evaluated people.

Kendall’s Tau: As a non-parametric correlation analysis method, if the relationship between multi-class and continuous variables does not have a correlation because the correlation coefficient does not exceed 0.6, it is determined as an invalid variable for emotional intelligence group classification. However, a new p-value threshold is set to prevent the p-value from being lowered due to multiple comparisons rather than the actual inter-class correlation. The p-value threshold is a false discovery rate (FDR), and FDR = false positive / total positive. That is, it is determined by adjusting the probability of not actually being significant among those determined to be significant, thereby correcting the significance (p-value). A Bonferroni correction may be applied to this correction.

Kruskal-Wallis test: an analysis method to determine non-parametric significance and report whether there is a difference between emotional intelligence groups and if there is no significant difference, it is determined as a variable that is not significant in emotional intelligence group classification. However, a new p-value threshold is set to prevent the p-value from being lowered due to multiple comparisons rather than the actual inter-class correlation. The p-value threshold is a false discovery rate (FDR), and FDR = false positive / total positive. That is, it is determined by adjusting the probability of not actually being significant among those determined to be significant, thereby correcting the significance (p-value). A Bonferroni correction may be applied to this correction.

After verifying the statistical validity of heart rate variability for emotional intelligence group classification by two methods such as the Kendall Tau and Kruskal-Wallis test, each effective variable of heart rate variability is obtained. Only the heart rate variability effective variable common to both analyzes of Kendall’s Tau and Kruskal-Wallis test is determined as the final effective variable. If only one test is significant, the variable is excluded from the valid variable.

### III.2. Emotional Intelligence Classification Criteria Heart Rate Variability Selection Step

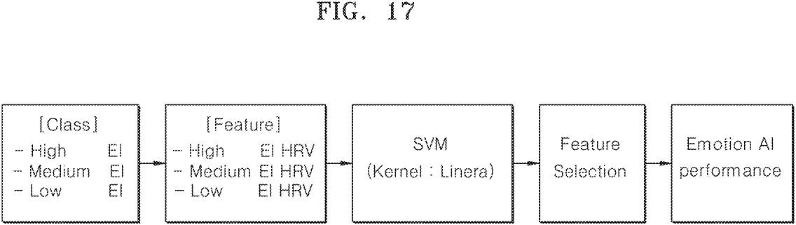

FIG. 17 shows the construction process of an artificial intelligence (AI) model for emotional intelligence level evaluation.

As shown in FIG. 17, an emotional intelligence level evaluation model is generated using a rule-based machine learning technique.

The emotional intelligence level classification class is divided into three classes (low emotional intelligence, average emotional intelligence, and high emotional intelligence), as shown in FIG. 15B.

The data used here is an effective variable for heart rate variability derived in the emotional intelligence classification criteria determination stage, and is used in a Train:Test ratio, that is, 0.8:0.2.

As a classification model in the inventive concept, a parameter of a kernel function to which a support vector machine (SVM) is applied is “linear”.

In Feature Selection, statistical analysis technique is used to determine the class of the input heart rate variability significant variable Feature, and the emotional intelligence level evaluation accuracy is derived by extracting the ranking of variables with high influence and selecting the model with the highest classification accuracy using various combinations of effective variables.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method of evaluating emotional intelligence, the method comprising:
a first evaluation step of presenting emotional image stimuli to a plurality of subjects through a display to evaluate an accuracy of emotional recognition for the emotional image stimuli;
a second evaluation step of presenting a facial image stimulus of a certain emotion to the subject to evaluate an accuracy of interpersonal emotional recognition of the subject;
a subject selection step of classifying subjects with high accuracy and low accuracy among the plurality of subjects according to results of the first evaluation step and the second evaluation step;
a video stimulus classification step of presenting a video of a certain emotion to the subject selected in the above process through the display to classify emotions of the video;
extracting a heart rate variability (HRV) from heartbeat information by a heartbeat information processing unit by extracting the heartbeat information of the subject exposed to a video of a certain emotion in the video stimulus classification step with a sensor;
forming a classification model by classification of the certain video stimulus and machine learning using the HRV through a model generating unit; and
evaluating an emotional intelligence of the subject exposed to the image stimulus using the classification model by an emotional intelligence evaluation unit.

2. The method of claim 1, wherein the certain emotion is classified into High Arousal and High Valence (HAHV), High Arousal and Low Valence (HALV), Low Arousal and Low Valence (LALV), and Low Arousal and High Valence (LAHV).

3. The method of claim 1, wherein the emotional image stimulus uses a photo stimulus presented by the International Affective Picture System (IAPS).

4. The method of claim 1, wherein the HRV comprises a time domain parameter.

5. The method of claim 4, wherein the time domain parameter comprises at least one of Heart Rate (HR), Standard Deviation of NN Interval (SDNN), and root Mean Square of successive differences (rMSSD) between intervals between peaks (PPI).

6. The method of claim 1, wherein the HRV comprises a frequency domain parameter.

7. The method of claim 6, wherein the frequency domain parameter of the HRV comprises at least one of high frequency (HF) power between 0.15 Hz and 0.04 Hz, very low frequency (VLF) power between 0.0033 Hz and 0.04 Hz, low frequency (LF) power between 0.04 Hz and 0.15 Hz, VLF/LF, LF/HF, total power, peak power, dominant power between 0.04 Hz to 0.26 Hz, and coherence ratio (peak power / (total power-peak power)).

8. The method of claim 1, wherein the classification model is a Super Vector Machine (SVM) classification model.

9. The method of claim 1, further comprising extracting effective HRV by evaluating a significance of heart rate variability based on an emotional intelligence evaluation score between the extracting of HRV and the forming of a classification model,
wherein the extracting of the effective HRV comprises performing nonparametric one-way analysis of variance (Kruskal-Wallis test) and Bonferroni correction to extract heart rate variability that is significantly different between groups by emotion, wherein for correlation comparison between emotional groups, Kendal-tau correlation analysis is performed to extract heart rate variability, which shows a correlation coefficient of 0.6 or higher.

10. The method of claim 9, wherein the classification model is a SVM classification model.

11. An emotional intelligence evaluation system for performing the method of claim 1, the emotional intelligence evaluation system comprising:
a display presenting video stimuli with certain emotions to a subject;
a heartbeat information extraction device equipped with a sensor for extracting heartbeat information from the subject;
a heart rate variability (HRV) extraction unit configured to extract HRV from the heartbeat information;
a model formation unit configured to form an emotional intelligence evaluation model using the HRV; and
an emotional intelligence evaluation unit configured to evaluate the subject’s emotional intelligence by applying the HRV of the subject exposed to a certain image stimulus, by using the model obtained from the model formation unit.

12. The emotional intelligence evaluation system of claim 11, wherein the heartbeat information extraction device comprises an electroencephalography (EEG) sensor or a photoplethysmography (PPG) sensor.

13. The emotional intelligence evaluation system of claim 11, wherein the image stimulus comprises a picture stimulus presented by the International Affective Picture System (IAPS).

14. The emotional intelligence evaluation system of claim 11, wherein the HRV comprises a time domain parameter.

15. The emotional intelligence evaluation system of claim 14, wherein the time domain parameter comprises at least one of Heart Rate (HR), Standard Deviation of NN Interval (SDNN), and root Mean Square of successive differences (rMSSD) between intervals between peaks (PPI).

16. The emotional intelligence evaluation system of claim 11, wherein the HRV comprises a frequency domain parameter.

17. The emotional intelligence evaluation system of claim 16, wherein the frequency domain parameter of the HRV comprises at least one of high frequency (HF) power between 0.15 Hz and 0.04 Hz, very low frequency (VLF) power between 0.0033 Hz and 0.04 Hz, low frequency (LF) power between 0.04 Hz and 0.15 Hz, VLF/LF, LF/HF, total power, peak power, dominant power between 0.04 Hz to 0.26 Hz, and coherence ratio (peak power / (total power-peak power)).
